(19) United States
(12) Patent Application Publication
(10) Pub. No.: US 2022/0000675 A1
(43) Pub. Date: Jan. 6, 2022

(54) CESAREAN SECTION INCISION SHELL

(71) Applicant: LANSINOH LABORATORIES, INC., ALEXANDRIA, VA (US)

(72) Inventors: Katherine Anne Bourdillon, Leeds (GB); Rush Lloyd Bartlett, II, Austin, TX (US); Kirsten Georgia Granger, Kirsten (GB)

(21) Appl. No.: 17/305,197

(22) Filed: Jul. 1, 2021

Related U.S. Application Data
(60) Provisional application No. 63/048,392, filed on Jul. 6, 2020.

(51) Int. Cl. A61F 13/14; A61F 13/02

(52) U.S. Cl. CPC A61F 13/145; A61F 2013/00255; A61F 13/0243; A61F 13/0246

(57) ABSTRACT

An example device for facilitating healing of a Cesarian section incision on a patient can include: a base configured to attach to skin of the patient, at least partially surrounding the Cesarian section incision; and a dome coupled with the base and extending over the Cesarian section incision, wherein the dome is configured to resist collapse when the device is worn under clothing.

### BACKGROUND

A Cesarean section (or “C-section”) is a surgical procedure used to deliver a baby through an incision in the lower abdomen and a second incision in the uterus. As the stitched surgical incisions heal, they can be painful, itchy and sore. Additionally, the location of the incision on the abdomen makes the incision prone to infection, dehiscence and complications caused by friction of clothing. Current management of C-section incisions during the initial healing process involves the use of standard surgical dressings or silicon sheets to protect the incision site and allow healing to occur. The location of the incision, however, often makes standard dressings uncomfortable and difficult to conform to the body along the lower abdomen. Traditional surgical dressings also rely on adhesives to secure them in place, which can cause pain on removal, particularly due to body hair typically found around a C-section incision. Silicone sheet dressings have a lower level of adhesive stickiness, but the adhesive is uniform across the entire sheet surface, which also often causes pain when removing in the area directly over the incision when sticking to the site.

Therefore, it would be desirable to have an improved device for protecting a Cesarean section incision from friction and clothing and thus facilitating healing. Ideally, such a device would protect the incision without directly contacting the incision.

### SUMMARY

In one aspect of the present disclosure, a C-section protective dressing (or “shell”) includes a molded silicone shell with a flat base and a semi-rigid dome to protect a C-section incision, allowing it to heal without directly contacting it. The C-section protective dressing helps relieve postpartum abdominal pain and discomfort, while reducing the risk of bacterial contamination and subsequent infection.

In some embodiments, the base is made of soft flexible silicone, which lies flat on the skin, ensuring good contact with the skin around the incision. The rigid dome includes a sidewall and provides protection of the incision from friction or irritation by clothing, while allowing air to circulate inside the dome. Air circulation helps with the removal of moisture or stale air in later stages of healing. It also protects any topical antimicrobial or medicinal agent applied to the incision from being rubbed off. In some embodiments, the dome may have perforations to further facilitate air circulation. Optionally, the shell may be combined with a secondary adhesive dressing to secure it in place.

These and other aspects and embodiments are described in greater detail below, in relation to the attached drawing figures.

### BRIEF DESCRIPTION OF THE DRAWINGS

FIG. 1 is a lateral perspective view of a Cesarean section incision shell, according to one embodiment;

FIG. 2 is a medial perspective view of the Cesarean section incision shell of FIG. 1;

FIG. 3 is a medial perspective view of a Cesarean section incision shell, with perforations to allow air circulation, according to another embodiment;

FIG. 4 is a top view of the Cesarean section incision shell of FIG. 3;

FIG. 5 is a top view of a Cesarean section incision shell with compression elements, according to another embodiment; and

FIG. 6 is a top view of a Cesarean section incision shell with a curved contour to enhance fit against the belly, according to another embodiment.

FIG. 7 is a perspective view of an example textile postpartum compression product holding the Cesarean section incision shell of FIG. 1.

FIG. 8 is another perspective view of the textile postpartum compression product of FIG. 7.

### DETAILED DESCRIPTION

The various concepts introduced above and discussed in greater detail below may be implemented in any of numerous ways, as the described concepts are not limited to any manner of implementation. Examples of specific implementations and applications are provided primarily for illustrative purposes.

The C-section dressing or shell described herein protects the incision area of a woman's abdomen post-Cesarian section, without directly touching the incision. This allows the incision to heal, relieving postpartum abdominal pain and discomfort, while reducing the risk of bacterial contamination and subsequent infection.

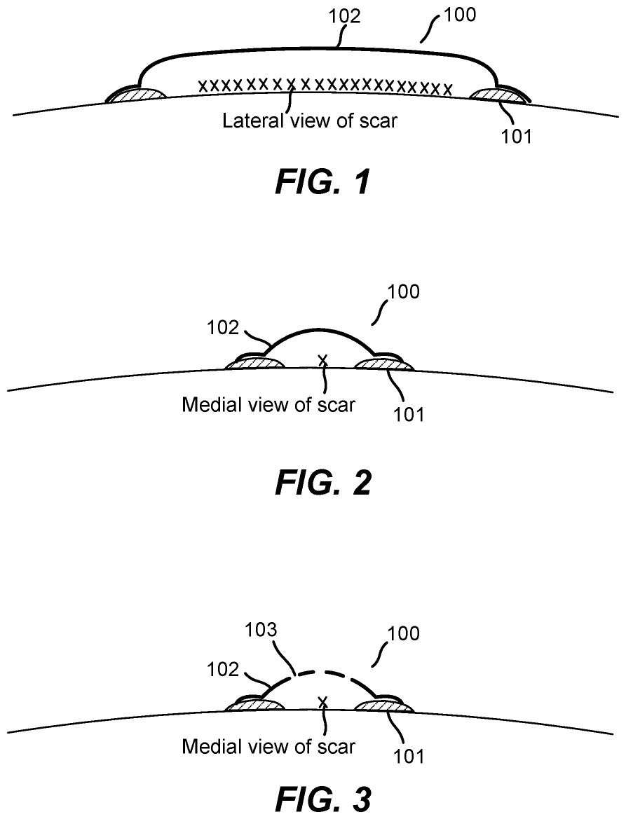

FIGS. 1 and 2 are perspective views, looking at the lateral and medial view of the incision respectively, of one example of a cesarean section incision shell 100, according to the present disclosure. In this embodiment, the shell 100 includes a flat base 101 and semi-rigid dome 102. The shell 100 may be constructed, for example, from molded silicone. The shell 100 may be contoured to fit the lower abdominal area comfortably while maintaining contact between the flat base 101 and the skin surrounding the incision.

The flat base 101 may be made of soft, flexible silicone, for example, which lies flat on the skin, ensuring good contact with the skin around the incision. In one embodiment, the flat base 101 extends approximately about 5.0 mm outward from the dome 102. In other embodiments, the flat base 101 may extend from about 0.1 mm to about 30 mm from the dome 102 of the shell 100. The skin-contact surface of the soft, flexible silicone of the flat base 101 may have a sufficient amount of inherent tack (or “stickiness”) to make the flat base 101 stay in position on, and adhere to, the skin, without using any additional adhesive agents. In one embodiment, the tacky silicone is uniform across the entire skin contact surface of the flat base 101. In some embodiments, the tack/stickiness of the skin-contact surface of the flat base 101 may be optimized by giving the surface a texture, pattern or dot-coating on either the entire surface or on sections. The tack is such that the flat base 101 can be moved and replaced on the skin while maintaining adhesive functionality. Alternatively or additionally, the flat base 101 may be coated with a secondary adhesive to facilitate attachment to the skin. Examples of such adhesives include, but are not limited to, acrylic, hydrocolloid, hydrogel, Polyurethane, and rubber adhesives. In some embodiments, such an adhesive may be coated onto a woven or non-woven material that is about 1-15% wider than the flat base 101 and is used as a secondary dressing to secure the C-section shell 100 in place.

In some embodiments, the semi-rigid dome 102 is also made of silicone. Alternatively, the semi-rigid dome 102 may be made of any number of semi-rigid polymers, such as but not limited to Polyurethane, polyether, PVC and PVA. Whatever material(s) is used, the semi-rigid dome 102 is constructed to resist compression by clothing and other materials and forces that might be applied to it during the activities of daily living.

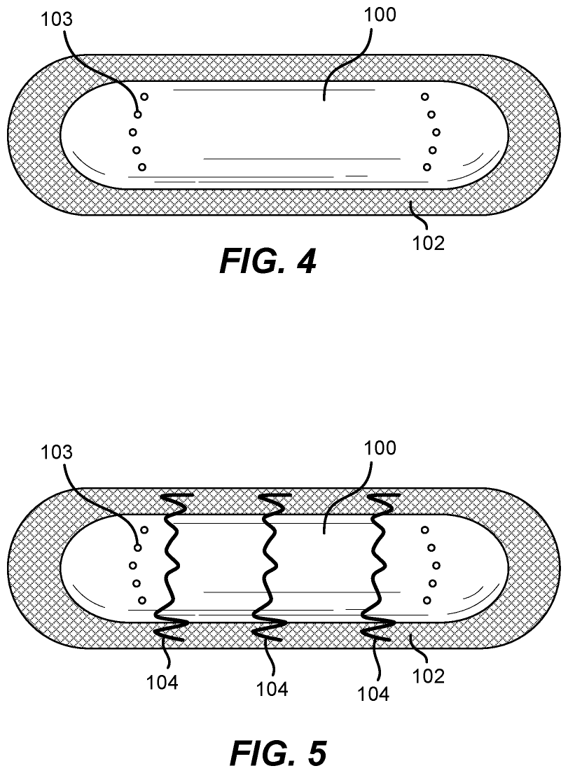

Referring now to FIGS. 3 and 4, an alternative embodiment of a C-section incision shell 100 is illustrated. In this embodiment, the semi-rigid dome 102 has multiple apertures 103. In alternative embodiments, the apertures 103 may be replaced by slits, perforations or any other suitably shaped and sized openings. The apertures 103 may be located at or near either end (or edge) of the dome 102, to facilitate circulation of air within the dome 102 when the shell 100 is secured on the skin. In various alternative embodiments, the apertures 103 may be uniformly or unevenly distributed across all or part(s) of the surface of the dome 102. Other embodiments may include any number, size, shape, pattern or configuration of apertures 103.

Referring now to FIG. 5, in another embodiment, a Cesarian section shell 100 may include a semi-rigid dome 102 with one or more compression elements 104, which add compression strength to the dome 102. This compression strength will apply pressure to hold the skin together, to relieve stress on the wound and reduce scar appearance or formation. The compression elements 104 may be made of metallic wire formed into a spring (or multiple springs) in some embodiments, such as but not limited to copper, steel, or aluminum. Alternatively, the compression elements 104 may be made of a shape memory material, such as but not limited to Nitinol. These compression elements 104 may be molded into spring tension and then applied by the user by flattening the shell 100 against the skin and then allowing it to compress back. Alternative embodiments, such as those with Nitinol compressive element(s) 104 may accomplish this spring motion with thermal actuation under body heat or high enough ambient heat. In alternative embodiments, the compression element(s) 104 may be generated by the way the semi-rigid dome 102 (silicone or other shell material) is molded and then made to compress by the user applying force to the dome 102 when applying the shell 100 to the skin and then cupping or compressing along the incision area to apply pressure to push it together.

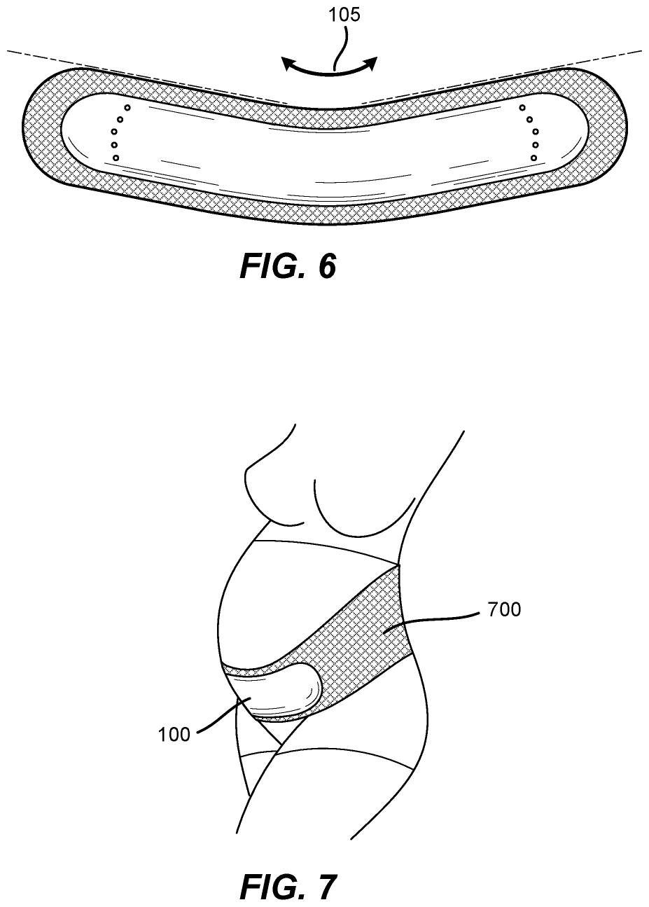

Referring now to FIG. 6, another embodiment of the C-section shell 100 is illustrated. In this embodiment, the dome 102 is has a curved contour 105, to allow it to curve around the belly where the incision is located. The curved contour 105 may be in any direction and have any angle or radius of curvature, according to various embodiments. In the embodiment of FIG. 6, the contour 105 curves laterally across the belly, but alternatively the contour 105 could extend radially, transverse, intersecting, vertical, or diagonal, for example. Whatever contour(s) 105 is/are used may help ensure that pressure on the wound is uniformly applied across the length of the wound. In further embodiments, the dome area of the shell is flattened and widened to give expanded coverage of the abdominal area

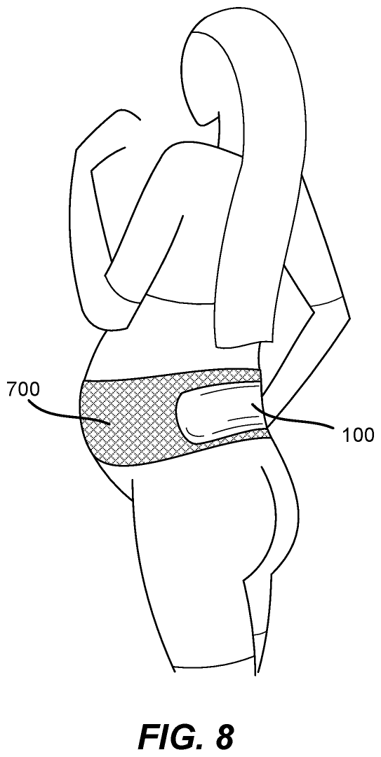

In a further embodiment shown in FIGS. 7-8, the C section shell 100 is secured in position through integration into a secondary textile device 700, including but not limited to a postpartum support band, maternity belt, nursing top, or high waisted underwear which holds the C section shell in place via gentle compression. The secondary textile device 700 could comprise a number of mechanisms to ensure securement of the shell in place around a torso of the patient. Those mechanisms may comprise but should not be limited to including nubs that fit into slots in the garment, a slide pouch for the shell to fit into that has an opening such that it would not touch that area of skin, a slide pouch for the shell to fit into that also touches skin with a breathable fabric or synthetic material, a set of snaps or buttons that enable removable connection of the shell to the garment inside segment from the top of the shell or sides of the shell, a magnetic element within the shell and or within the garment that would enable securement through magnetic attraction of one body to the other, temporary adhesive material on the outside of the shell or inside of the garment to attach one to the other, or the use of Velcro material on the shell and garment such that the securement could rest in place. In the embedment where the C-section shell is able to be slid into a pouch in the garment one embodiment could enable the garment to have elastic bands that are wrapped around the shell prior to the garment being applied to a user. Those elastic bands could secure the shell to the garment.

Similarly, a pouch with or without an internal opening to create airspace against the skin when the shell is inserted could be used within the garment such that the pouch was opened and the shell was inserted and then the shell was closed to enable securement where the shell rests inside a pouch and gives the pocket of the pouch a ridged or semi-ridged shape while the garment closure, wrap, or other mechanism such as Velcro or snaps are used to secure the top outer portion of the garment closure to the pouch. In other such embodiments the compartment or position where a c-section structural support shell is used within a garment could be made such that there could also be room for a hot/cold pack element. This hot or cold pack element could enable the shell or the surrounding area to provide cooling or heating to that area.

Similarly in another such embodiment the garment could be made to be reversible such that the hot or cold pack element in the pocket, groove, slot, or other mechanism for positioning of an element of hot or cold pack or shell could be made into the position on the back instead of on the c-section shell area. See FIG. 8. In other such embodiments, two pouches, slots, guide areas, or other mechanisms could be constructed so that the C-section support shell could be used in the front and a hot and or cold pack could be used on the back. Alternatively, additional embodiments could comprise multiple pouches, slots, or guides where hot and cold packs could be placed laterally, in the front, in the back, or otherwise in combination with a support shell in the front that is removable and or integrated directly within the structural frame of a compression support garment such that the garment compression does not contact at least a portion of the skin near or on top of the C-section incision or if it does contact at least the compression garment acts to dissipate the strongest force away from the incision line itself to other areas adjacent to it on the belly or other parts of the body.

In any of the above-described embodiments, the C-section shell may be combined with a topical application of one or more therapeutic substances for example in the following therapeutic uses: treatment of pain, healing of tissue, anti-itch, postpartum depression, reduction of infection risk, swelling and or bleeding reduction, and other therapeutic benefits as prescribed and delivered to the site of care by the device construction disclosed herein. These therapeutically active ingredients may include, but are not limited to, anti-inflammatory or pain relieving drugs (ibuprofen or NSAIDs, lidocaine, benzocaine, acetocaine, tetracaine, prilocaine, menthol, morphine, oxycodone, acetaminophen, ethanol, cannabinoids/cannabis, hemp oils, paracetamol, codeine, Nefopam, Flupirtine, Ziconotide), anticonvulsant drugs (carbamazepine, gabapentin, pregabalin, gabapentinoids, and others including those used to treat neuropathic pain), vasoconstrictors (pseudoephedrine, epinephrine, Phenylephrine), anti-depressants (tricyclic antidepressants, lithium etc.), vitamins and their derivatives (vitamin D2 and D3, vitamin C, ascorbyl palmitate, ascorbic acid, vitamin E, argan oil), antimicrobials (Silver, iodine, chlorhexidine, PHMB, Quaternary Ammonium compounds, anti-fungal agents), other anti-inflammatory agents (capsaicin, resveratrol), wound healing promotors (hydrogel, Carboxymethyl cellulose, Collagen, Collagen-ORC) and any other desired material that has a therapeutic effect that may be achieved through local delivery of the therapeutically active material to the surface of the incision site.

This is believed to be a complete and accurate description of embodiments and aspects of the invention. Alternative embodiments are contemplated within the scope of the invention, however, and the above description is intended for exemplary purposes and not to limit the scope of the invention as described, for example, in the following claims.

We claim:

1. A device for facilitating healing of a Cesarian section incision on a patient, the device comprising:
a base configured to attach to skin of the patient, at least partially surrounding the Cesarian section incision; and
a dome coupled with the base and extending over the Cesarian section incision, wherein the dome is configured to resist collapse when the device is worn under clothing.

2. The device of claim 1, wherein the base is made of soft, flexible silicone.

3. The device of claim 1, wherein the base surrounds the dome and extends approximately about 5.0 mm outward from the dome.

4. The device of claim 1, wherein a skin contact surface of the base comprises a texture.

5. The device of claim 1, wherein a skin contact surface of the base comprises an adhesive.

6. The device of claim 1, wherein the dome is made of a material selected from the group consisting of silicone, polyurethane, polyether, PVC and PVA.

7. The device of claim 1, wherein the dome comprises multiple apertures to facilitate air circulation through the dome.

8. The device of claim 7, wherein the multiple apertures comprise:
a first set of apertures near a first end of the dome; and
a second set of apertures near a second end of the dome.

9. The device of claim 1, wherein the dome is integrated into a textile postpartum compression product.

10. The device of claim 9, wherein the textile postpartum compression product is a support band.

11. The device of claim 10, wherein the support band is sized to fit around a torso of the patient.

12. The device of claim 1, wherein the base and the dome include a curve.

13. The device of claim 1, wherein the dome comprises one or more compression members.

14. A method of facilitating healing of a Cesarian section incision on a patient, the method comprising:
applying a shell dressing over the Cesarian section incision, the shell dressing comprising:
a base configured to attach to skin of the patient, at least partially surrounding the Cesarian section incision; and
a dome coupled with the base and extending over the Cesarian section incision,
wherein the dome is configured to resist collapse when worn under clothing, and
wherein when the shell dressing is applied to the patient, neither the base nor the dome contacts the Cesarian section incision.

15. The method of claim 14, further comprising promoting air circulation through the dome of the shell dressing by providing the dome with multiple apertures.

16. The method of claim 15, wherein the multiple apertures comprise:
a first set of apertures near a first end of the dome; and
a second set of apertures near a second end of the dome.

17. The method of claim 14, further comprising applying a therapeutic agent to the Cesarian section incision before applying the shell dressing.

18. The method of claim 14, wherein the dome is integrated into a textile postpartum compression product.

19. The method of claim 18, wherein the textile postpartum compression product is a support band.

20. The method of claim 19, wherein the support band is sized to fit around a torso of the patient.
